(19) United States
(12) Patent Application Publication
(10) Pub. No.: US 2021/0128015 A1
(43) Pub. Date: May 6, 2021

(54) SYSTEM AND METHOD FOR ACTIVE CANCELLATION FOR PRESSURE PULSES

(71) Applicant: KONINKLIJKE PHILIPS N.V., EINDHOVEN (NL)

(72) Inventor: Eugene Peter Gerety, Seymour, CT (US)

(21) Appl. No.: 16/491,588

(22) PCT Filed: Mar. 12, 2018
(86) PCT No.: PCT/EP2018/055991

§ 371 Date: Sep. 6, 2019

Related U.S. Application Data
(60) Provisional application No. 62/472,963, filed on Mar. 17, 2017.

(51) Int. Cl. A61B 5/08; A61B 5/00; A61B 5/087; A61B 5/097

(52) U.S. Cl. CPC A61B 5/082; A61B 5/0803; A61B 5/7203; A61B 2562/0247; A61B 5/087; A61B 5/097; A61B 5/0082

(57) ABSTRACT

A respiration gas monitor device (100) includes a pump (110) connected to draw a flow of respired air, a pressure sensor (150, 160) is connected to measure an air pressure signal responsive to the flow of respired air, and a pressure transducer (180c). Electrical circuitry (170, 180) is operatively connected to measure flow across the pressure sensor. A gas component sensor (190, 192, 194) is arranged to monitor a target gas in the flow of respired air.

### FIELD

The following relates generally to the monitoring arts, respiration arts, pressure pulsation monitoring arts, pressure pulsation cancellation arts, gaseous concentration measurement arts, and related arts.

### BACKGROUND

In sidestream respiration gas monitors (RGMs), also known as diverting RGMs, a sample of respiration gas is drawn from a patient down a sample tube to a measuring area of the RGM (respiration gas monitor) where any of a variety of techniques can be used to measure the concentration of one or more components of the respiration gas including, but not limited to, carbon dioxide (CO₂), oxygen (O₂), nitrous oxide (N₂O), and halogenated agents such as halothane, enflurane, isoflurane, sevoflurane and desflurane. Patterns of variation in the concentrations of these gas components can have clinical significance in the treatment of patients. Accordingly, it is desired to provide a consistent, accurate temporal record of the monitored gas concentration to aid in the diagnosis and treatment of a variety of conditions. To this end, the manner of gas sampling can have a great influence on the performance and accuracy of an RGM.

Typically, a small diaphragm pump or similar air-moving device is used to create the gas flow from the patient to the measuring area. By the reciprocating nature of their operation, such pumps tend to move the sample gas in a pulsatile manner, producing significant pressure variation, i.e. ripple, in the sample line, especially in the vicinity of the pump. Other types of mechanical air pumps similarly tend to introduce a pressure ripple due to the cyclical nature of the pumping mechanism, usually at the cycle frequency or a multiple thereof, e.g. twice the cycle frequency. These pressure variations, depending upon their magnitude and frequency, can interfere with flow rate measurement and gas concentration measurement.

Two methods that have been used previously to deal with these pressure pulsation (i.e. ripple) include: (1) providing an air “reservoir” to absorb and attenuate the pulsation, effectively reducing the amplitude of the pulsations to manageable levels; and (2) low-pass filtering the pressure-drop measurement to attenuate the pulsation. Both of these methods have disadvantages. The reservoir approach, while effective, adds significant physical volume, which is disadvantageous where space is at a premium, and there is significant desire to reduce the physical volume of RGMs.

The low-pass filtering approach adds little physical volume, but does nothing to attenuate or eliminate the pressure pulsation in the gas sampling system. Since the pulsations can be very large compared to the pressure drop associated with flow, this creates a risk of pressure sensor saturation unless a sensor with a very wide pressure sensing range is employed. If the sensor saturates, the low-pass method produces an error in flow rate measurement, but selecting a wide-range sensor (compared to the desired measurement) usually results in poor measurement accuracy unless a very expensive, high-accuracy sensor is chosen. Further, this technique permits the un-attenuated pressure pulsations to appear in the measurement area, producing errors in the measurement of gas concentrations.

Improvements disclosed herein address the foregoing and other disadvantages of respiratory gas monitoring systems, methods, and the like.

### BRIEF SUMMARY

In accordance with one illustrative example, a respiration gas monitor device includes a pump connected to draw a flow of respired air, a pressure sensor is connected to measure an air pressure signal responsive to the flow of respired air, and a pressure transducer. Electrical circuitry is operatively connected to measure flow across the pressure sensor. A gas component sensor is arranged to monitor a target gas in the flow of respired air.

In accordance with another illustrative example, a device for attenuating or eliminating pressure ripple in a respiration gas monitor is provided. The device includes a pump configured to draw respired air from a measurement area, and a constrictor through which at least a portion of the respired air drawn by the pump moves. At least one pressure sensor is configured to measure a pressure value of air flowing through the constrictor. A ripple cancellation device is configured to attenuate or eliminate at least one pressure ripple in the respired air flowing through the constrictor.

In accordance with another illustrative example, a respiratory gas monitoring method includes: drawing, with a pump, respired air through a measurement area, at least a portion of the respired air moving through a constrictor; measuring, with at least one pressure sensor, a pressure signal of air flowing through the constrictor; attenuating or eliminating, with a ripple cancellation device, at least one pressure ripple in the respired air flowing through the constrictor; and measuring, with a measurement device, a target gas in the flow of expired air.

One advantage resides in removing pressure pulsations in an air pressure signal.

Another advantage resides in measuring gases in air without pressure pulsations.

Another advantage resides in controlling flow in a pump by removing pressure pulsations.

Another advantage resides in measure concentrations of different gases in respired air.

Further advantages of the present disclosure will be appreciated to those of ordinary skill in the art upon reading and understand the following detailed description. It will be appreciated that a given embodiment may provide none, one, two, or more of these advantages.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a respiration gas monitor device according to one aspect.

FIG. 2 is an exemplary flow chart of the calibration process of the device of FIG. 1.

### DETAILED DESCRIPTION

In RGMs, the respiration gas flow is typically regulated to a relatively constant rate to avoid temporal distortion of the gas concentration record (i.e., waveform). This flow regulation is often accomplished by introducing a constrictor (such as an orifice or a capillary tube) into the gas flow path and controlling the pump drive level is controlled to maintain a constant pressure drop across the constrictor. Since the pressure drop is a direct function of the flow rate, maintaining a constant pressure drop produces a constant flow rate. In the presence of pump-induced pressure pulsations, however, the amplitude of the pulsations in the pressure drop can be large, and may even exceed the magnitude of the flow-induced pressure drop, which can be problematic for accuracy of the flow rate measurement and control.

Another problem potentially introduced by pressure pulsations in the sample line is that the pressure pulsations appear on the gas sample in the measuring area of the RGM. The gas concentration measurements can be affected by the temperature and pressure of the gas in the measuring area, so any significant pressure pulsations can interfere with the accuracy of those measurements.

The following relates to an improvement in Respiration Gas Monitor (RGM) devices employing a sidestream (i.e. diverting) flow arrangement in which a pump is provided to draw sample gas from the main respiration circuit into a side stream feeding the RGM device. The pump produces a negative pressure (a “vacuum”) that draws the flow into the side stream. A diaphragm pump is commonly used, which produces a constant or average negative pressure on which is superimposed a “ripple” pressure variation component. The average negative pressure can be on the order of 1 psi while the ripple may be comparable, e.g. 0.5 psi. Other types of pumps also operate using a cyclical pumping cycle that similarly typically introduces a pressure ripple. This ripple introduces pressure variations in the sampling chamber. Since the CO2 or other gas measurement is pressure-dependent, the ripple can introduce respiration gas measurement errors.

The following describes improved RGM devices in which an audio transducer is provided to reduce or eliminate the pressure ripple introduced by the pump. In some illustrative embodiments, the transducer control circuit includes a differential pressure sensor to measure the pressure drop over a constrictor (i.e., orifice or capillary tube) and a high-pass or bandpass filter to filter the measured pressure to extract the ripple. The high-pass filtered signal is inverted and applied to drive the audio transducer to generate an opposing ripple that reduces or cancels the ripple produced by the pump. The transducer is thus used to provide a more uniform pressure output from the fluid pump.

There are many ways of sensing flow, including but not limited to: hot-wire, ultrasonic sensing by differential time delay, and measuring the pressure drop across an obstacle in the flow path, among others. The following describes the pressure drop/obstacle method, but other flow sensing techniques could be adapted. With the pressure drop/obstacle method of flow sensing, a “constrictor” device is used. The constrictor is essentially any obstacle placed in the flow path. The obstruction to flow produces a relative drop in pressure on the “lee” side of the obstacle when air (gas) passes by it. This difference in pressure is a function of flow. The pressure sensor measures the difference in pressure before and after the obstruction, thereby producing an electrical signal responsive to and representative of airflow past the obstacle.

The most common constrictor type is an orifice. The orifice is very inexpensive, but has the disadvantage that it is highly nonlinear and highly sensitive to temperature and various gas properties. Another type of constrictor is a capillary tube, which is highly linear and much less sensitive to temperature and other sources of error. The capillary tube, used in this way, is sometimes referred to as a “linear flow converter”.

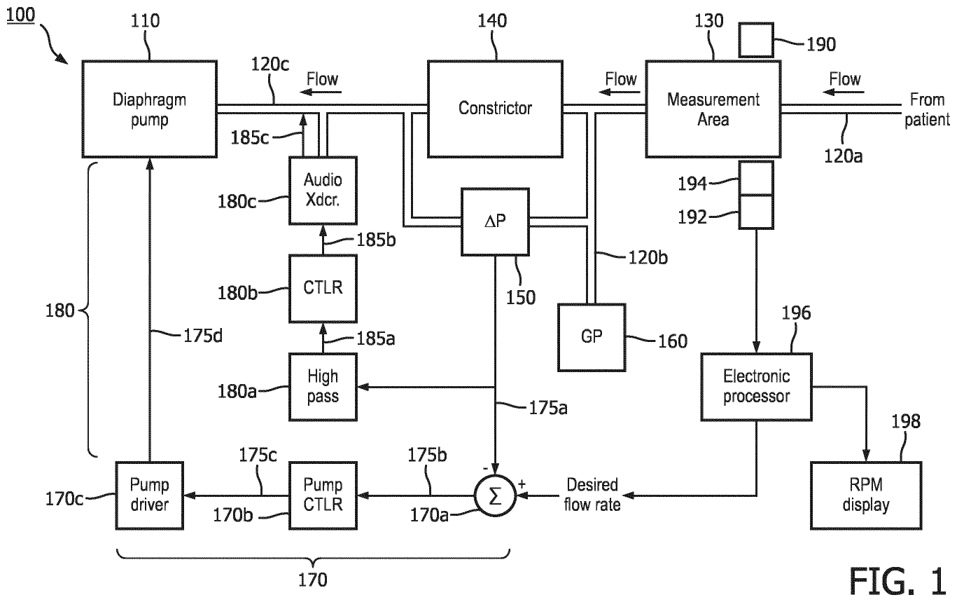

With reference now to FIG. 1, a schematic illustration of a respiratory gas monitor (RGM) device 100 including components for attenuating or eliminating a pressure ripple. The RGM device 100 includes an air-moving device, such as a diaphragm pump 110, connected to draw a flow of respired air by a patient. In a typical sidestream configuration, the respired air is drawn from a nasal cannula, tracheal intubation, or other patient accessory. The RGM device 100 also includes a flow constrictor 140 through which at least a portion of the respired air drawn by the pump moves. The pump 110 is connected to draw a flow of respiration gas from a patient through a sample tube segment 120a into a measurement area 130. The air then flows to the constrictor 140, which is disposed between the pump 110 and the measurement area 130. The pump 110 then receives the air from the constrictor 140. Ultimately, the air drawn by the pump 110 may be discharged to the ambient air, optionally after passing through a scrubbing device (not shown). A sample tube segment 120b provides connection of a differential pressure sensor 150 to measure differential pressure across the constrictor 140, and connection of an optional gauge pressure sensor 160. In some examples, the constrictor 140 can be a capillary tube or an orifice.

The device 100 also includes the at least one pressure sensor 150 connected to measure an air pressure signal responsive to the flow of respired air through the constrictor 140. For example, the flow of respiration gas through the constrictor 140 creates a pressure drop across the constrictor. In this example, the pressure sensor 150 is a differential pressure sensor that measures the pressure decrease from the inlet to the outlet of the constrictor 140. The pressure sensor 150 is configured to measure this pressure drop and produce a differential pressure signal 175a representative of the pressure drop across the constrictor 140, (and hence representative of the rate of gas flow through the constrictor). In some examples, the pump 110 is connected to draw the flow of respired air through the constrictor 140 and the pressure sensor 150 is connected to measure the air pressure signal indicating a pressure change across the constrictor. The constrictor 140 and the pressure sensor 150 serve to maintain a constant rate of airflow. A control mechanism, such as a pump controller (not shown) drives the pump 110 to maintain a constant pressure drop across the constrictor (as measured by the differential pressure sensor). The device 100 optionally further includes a gauge sensor 160 arranged to monitor pressure of the respired air. The gauge pressure sensor 160 is configured to measure the pressure at the outlet of the measurement area 130, and hence represents pressure of the respired air in the measurement area 130. This gauge pressure measurement is optionally used in the calculation of the concentrations of the target gas (e.g. carbon dioxide in the case of the RGM device 100 being a capnometer) in the respiration gas sample present in the measurement area 130 at any given time. In the illustrative example, a target gas measurement device is an optical measurement device that includes an infrared light source 190, a light detector 192, and a bandpass filter 194. The infrared light source 190 is arranged to launch infrared light that is transmitted through the measurement area 130, and more particularly through the flow of respired air through the measurement area 130. The bandpass filter 194 is arranged to filter the infrared light to pass a wavelength absorbed by the target gas (that is, the bandpass filter 194 has a passband that encompasses an absorption line of the target gas, e.g. the 4.3 micron absorption line of carbon dioxide in the case of a capnometer). The light detector 192 is arranged to detect the infrared light after being transmitted through the flow of respired air and filtered by the bandpass filter 194. The target gas concentration or partial pressure is computed, e.g. by a microprocessor or other electronic processor 196, based on the detected infrared light intensity. A higher concentration of the target gas in the respired air produces more absorption and hence a reduced transmitted and bandpass-filtered infrared light intensity. Optionally, the determination of the concentration or partial pressure of target gas takes into account known factors that can affect the measurement, such as the pressure of the respired air as measured by the gauge pressure sensor 160, and/or a calibration infrared intensity measured in the absence of the respired air flow. The electronic processor 196 may also optionally compute a clinically significant value, such as end-tidal carbon dioxide (etCO₂) in the case of the RGM device 100 implementing capnography. The target gas measurement and/or derived clinical value such as etCO₂ is displayed on an RGM display 198 (e.g. an LCD display showing the target gas concentration or partial pressure and/or the derived clinical quantity as a real-time numeric value, and/or as a trend line, or so forth). Additionally or alternatively, the data may be ported off the RGM device 100 via a wired or wireless communication link (not shown, e.g. a wired or wireless Ethernet link, a Bluetooth link, et cetera). The electronic processor 196 may also optionally perform various RGM device control functions, such as outputting the desired flow rate to the flow control mechanism 170.

The illustrative optical target gas measurement device 190, 192, 194 is merely an illustrative example, and more generally any type of target gas measurement device may be employed to measure the concentration or partial pressure of the target gas in the respired air flowing through the measurement area 130.

In some examples, the pump 110 is a reciprocating or cyclically operating device that moves air (i.e., respiration gas) in a pulsatile fashion, thereby producing significant pressure pulsations (i.e. pressure ripple) in the tubing segment 120c. If these pressure pulsations are transmitted via the constrictor 140 and sample tube segment 120b to the measurement area 130, then they can lead to measurement error. The amplitude of the pulsations is likely to be reduced somewhat after passing through the tubing segments 120c, 120b and the constrictor 140, but this attenuation may not be enough to prevent a significant pulsation waveform to appear in measurements made by the gauge pressure sensor 160. These pulsations can create errors in the measured concentrations of the components of the respiration gas sample present in the measurement area 130.

The device 100 can also include electronic circuitry configured to control various operations thereof (e.g., flow control, pulse cancellation, and the like). To control flow operations, the electrical circuitry of the device 100 can include a flow control mechanism 170 with a comparator 170a, a pump controller 170b, and a pump driver 170c arranged in a feedback control configuration. The comparator 170a is configured to receive the differential pressure signal 175a from the differential pressure sensor 150. From this, the comparator 170a is configured to subtract the differential pressure signal 175a (i.e., the flow rate through the constrictor 140) from a desired flow rate setpoint signal to produce or generate a flow rate error signal 175b indicative of the difference between the desired flow rate and the actual flow rate. The pump controller 170b is configured to amplify and process the flow rate error signal 175b to produce or generate a pump control signal 175c, which is used to driver a pump driver 170c. The pump driver 170c is configured to buffer the pump control signal 175c to produce or generate a pump drive signal 175d, which is transmitted to the pump 110 and used to drive the pump. If the differential pressure signal 175a indicates that the flow rate is less than the desired flow rate, the resultant error signal 170b indicates that the flow rate should be increased by increasing the speed of the pump 110. When this occurs, the pump driver 170c is configured to increase the speed of the pump 110. Conversely, if the differential pressure signal 175a indicates that the flow rate is greater than the desired flow rate, the resultant error signal 170b indicates that the flow rate should be decreased by decreasing the speed of the pump 110. When this occurs, the pump driver 170c is configured to decrease the speed of the pump 110. The pump controller 170b is configured to control the pump 110 in a manner that will produce a stable, steady flow rate. Other types of feedback control of the pump 110 are contemplated. It is further contemplated to operate the pump 110 without feedback control, i.e. in open loop fashion.

As disclosed herein, a closed-loop control ripple cancellation device 180 is provided to cancel and thereby reduce or eliminate the pressure ripple introduced by the cyclical operation of the pump 110. To provide pressure pulsation cancellation, the device 100 includes a pressure transducer 180c (or other suitable device) which introduces a pressure ripple that is “opposite” that produced by the pump 110, so as to cancel the pressure ripple of the pump 110. The electronic circuitry 180a, 180b is operatively connected to read the pressure sensor 150 and to drive the pressure transducer 180c to inject ripple-canceling pressure pulses into flow of respired air to reduce or eliminate a pressure ripple in the flow of respired air. The ripple-canceling pressure pulses are determined by the electrical circuitry 180a, 180b from the air pressure signal measured by the pressure sensor 150. In other contemplated embodiments, gauge pressure measured by the gauge pressure sensor 160 is used, and those ripples are controlled instead. This may have an advantage in placing the ripple control driver closer to the capnography sensor. Cancellation of the pressure pulsations is accomplished with a closed-loop control ripple cancellation device 180 configured to attenuate or eliminate at least one pressure ripple in the respired air flowing through the constrictor 140. The illustrative ripple cancellation device 180 includes a high-pass filter 180a, a controller 180b and the audio transducer (or similar air moving device) 180c. An AC component of the differential pressure signal 175a measured by the differential pressure sensor 150 is representative of the pressure pulsations created by the pump 110. The high-pass filter 180a is configured to receive the pressure value from the pressure sensor 150 and separate out and isolate this AC component of the signal to generate such that a pulsation or ripple signal 185a that represents only those pulsations, without regard to a flow rate related component of the differential pressure signal 175a. The cut-off frequency of the high-pass filter 180a is chosen to pass the AC component corresponding to the pressure ripple. It will be appreciated that the high-pass filter 180a may be replaced by a bandpass filter whose lower and upper cut-off frequencies are chosen such that the ripple signal is within the passband. On the other hand, the (lower) cut-off frequency of the bandpass filter 180a should be high enough to remove the DC pressure component, so that the output of the filter 180a corresponds to the pressure ripple component alone. The controller 180b is programmed or tuned to produce or generate a transducer drive signal 185b from the ripple signal to drive the audio transducer 180c. The audio transducer 180c produces or generates an antiphase pressure waveform 185c from the transducer drive signal 185b that counteracts and substantially nullifies the pressure pulsations (ripple) produced by the pump 110, the goal of which being to produce minimal signal output from the high pass filter 180a. The transducer 180c is configured to apply the antiphase pressure waveform to air flowing from the constrictor 140 to the pump 110 to nullify the pulsations in the air. In some embodiments, the controller 180b is a proportional-integral-derivative (PID) controller having proportional (P), integral (I), and derivative (D) parameters. The PID controller may be implemented using analog circuitry (e.g. op amps) and/or digital circuitry, e.g. a microprocessor or microcontroller. The controller 180b can also be some other type of feedback controller (e.g. a PI controller).

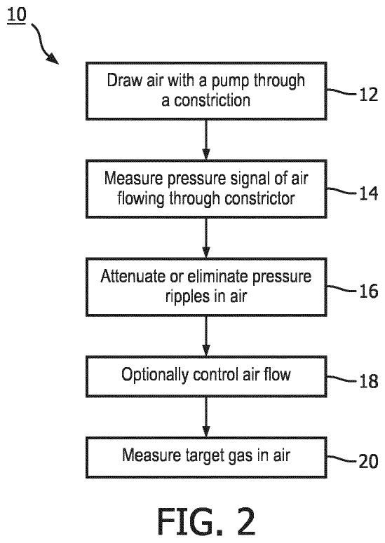

With reference now to FIG. 2, the RGM device 100 is configured to perform a respiratory gas monitoring method 10. At 12, respired air is drawn, with the pump 110, through the measurement area 130. At least a portion of the respired air moves through the constrictor 140. At 14, a pressure signal 175a of air flowing through the constrictor 140 is measured with the at least one pressure sensor 150.

At 16, at least one pressure ripple in the respired air flowing through the constrictor 140 is attenuated or eliminated with the ripple cancellation device 180a, 180b, 180c. The attenuation or elimination includes separating, with the filter 180a, an AC component of the pressure signal to generate a ripple signal 185a. A transducer drive signal 185b is generated from the ripple signal 185a with the controller 180b. An antiphase pressure waveform 185c is generated from the transducer drive signal 185b with the pressure transducer 180c. The waveform 185c is then applied by the transducers 180c to air flowing from the constrictor 140 to the pump 110 to nullify the pulsations in the air. It should be noted that as the antiphase pressure waveform 185c cancels the pressure ripple produced by the pump 110 within the sample tube segment 120c, this cancellation also removes the pressure ripple produced by the pump 110 for all points “upstream” of the sample tube segment 120c, particularly in the constrictor 140 and the further-“upstream” measurement area 130.

At 18, a flow of the air is optionally controlled with the flow control mechanism 170a, 170b, 170c. (Note that operations 16 and 18 are performed concurrently). To do so, the comparator 170a, is configured to subtract the pressure signal from a desired flow rate setpoint signal 175a to generate a flow rate error signal 175b. The pump controller 170b is configured to amplify and process the flow rate error signal 175b to produce or generate a pump control signal 175c, which is used to drive a pump driver 170c. The pump driver 170c is configured to buffer the pump control signal 175c to produce or generate a pump drive signal 175d, which is transmitted to the pump 110 and used to drive the pump. When the differential pressure signal 175a indicates that the flow rate is less than the desired flow rate, the pump driver 170c is configured to increase the speed of the pump 110. When the differential pressure signal 175a indicates that the flow rate is greater than the desired flow rate, the pump driver 170c is configured to decrease the speed of the pump 110.

At 20, a target gas in the flow of expired air is measured with the gauge pressure sensor 160. (Again, operation 20 is performed concurrently with operations 16, 18).

Referring back to FIG. 1, the electrical circuitry of the device 100 (e.g., the flow control mechanism with a comparator 170a, the pump controller 170b, and the pump driver 170c, the high-pass filter 180a, and the controller 180b and the electronic processor 196) can be implemented as one or more microprocessors, microcontrollers, FPGA, or other digital device(s), and/or by analog circuitry.

It will be appreciated that the illustrative computational, data processing or data interfacing components of the device 100 may be embodied as a non-transitory storage medium storing instructions executable by an electronic processor (e.g., the electronic processor 196) to perform the disclosed operations. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

The disclosure has been described with reference to the preferred embodiments. Modifications and alterations may occur to others upon reading and understanding the preceding detailed description. It is intended that the disclosure be constructed as including all such modifications and alterations insofar as they come within the scope of the appended claims or the equivalents thereof.

1. A respiration gas monitor device, comprising:
a pump connected to draw a flow of respired air;
a pressure sensor connected to measure an air pressure signal responsive to the flow of respired air;
a pressure transducer;
electrical circuitry operatively connected to measure flow across the pressure sensor, wherein the electrical circuity is operatively connected to read the pressure sensor and to drive the pressure transducer to inject ripple-canceling pressure pulses into flow of respired air to reduce or eliminate a pressure ripple in the flow of respired air wherein the ripple-canceling pressure pulses are determined by the electrical circuitry from the air pressure signal measured by the pressure sensor;
and
a gas component sensor arranged to monitor a target gas in the flow of respired air.

2. The respiration gas monitor device according to claim 1, wherein the electrical circuitry determines the ripple-canceling pressure pulses by operations including high-pass or bandpass filtering the air pressure signal measured by the pressure sensor.

3. The respiration gas monitor device according to claim 1, further comprising a constrictor comprising a capillary tube or an orifice; wherein the pump is connected to draw the flow of respired air through the constrictor and the pressure sensor is connected to measure the air pressure signal indicating a pressure change across the constrictor.

4. The respiration gas monitor device according to claim 1, wherein the electrical circuitry includes one of a proportional-integral-derivative (PID) controller and a microprocessor.

5. The respiration gas monitor according to claim 1, wherein the gas component sensor includes:
an infrared light source arranged to transmit infrared light through the flow of respired air;
a bandpass filter arranged to filter the infrared light to pass a wavelength absorbed by the target gas, and
a light detector arranged to detect the infrared light after being transmitted through the flow of respired air and filtered by the bandpass filter.

6. The respiration gas monitor according to claim 1, wherein the pressure sensor is one of:
(i) a differential pressure sensor connected to measure differential pressure across a constrictor in a path of the flow of respired air; or
(ii) a gauge pressure sensor connected to measure a gauge pressure of the flow of respired air.

7. A device for attenuating or eliminating pressure ripple in a respiration gas monitor, the device comprising:
a pump configured to draw respired air from a measurement area;
a constrictor through which at least a portion of the respired air drawn by the pump moves;
at least one pressure sensor configured to measure a pressure value of air flowing through the constrictor and to measure a differential pressure signal of air flowing through the constrictor, the at least one pressure sensor including as differential pressure sensor disposed at each of an inlet and an outlet of the constrictor; and
a ripple cancellation device configured to attenuate or eliminate at least one pressure ripple in the respired air flowing through the constrictor, the ripple cancellation device further including:
a filter configured to receive the pressure value from the pressure sensor and to separate an AC component of the pressure signal to generate a ripple signal,
a controller configured to generate a transducer drive signal from the ripple signal, and
a pressure transducer configured to produce an antiphase pressure waveform from the transducer drive signal, and to apply the antiphase pressure waveform to air flowing from the constrictor to the pump to nullify the pulsations in the air.

8-11. (canceled).

12. The device according to claim 7, further including a flow control mechanism configured to control flow of air from the pump, the flow control mechanism including:
a comparator configured to receive the differential pressure signal from the differential pressure sensor, and subtract the differential pressure signal from a desired flow rate setpoint signal to generate a flow rate error signal;
a pump controller configured to amplify and process the flow rate error signal to generate a pump control signal; and
a pump driver configured to buffer the pump control signal to generate a pump drive signal, and transmit the pump drive signal to the pump.

13. The device according to claim 12, wherein
the pump driver is configured to increase the speed of the pump when the differential pressure signal is less than the desired flow rate setpoint signal, and.
the pump driver is configured to decrease the speed of the pump when the differential pressure signal is greater than the desired flow rate setpoint signal.

14. The device according to claim 7, wherein the pump is configured to draw air from a patient first through a measurement area and then through the constrictor, whereby the constrictor is disposed between the pump and measurement area.

15. (canceled).

16. A respiratory gas monitoring method, comprising:
drawing, with a pump, respired air through a measurement area, at least a portion of the respired air moving through a constrictor;
measuring, with at least one pressure sensor a pressure signal of air flowing through the constrictor;
attenuating or eliminating, with a ripple cancellation device, at least one pressure ripple in the respired air flowing through the constrictor, separating, with a filter, an AC component of the pressure signal to generate a ripple signal, generating, with a controller, a transducer drive signal from the ripple signal, and producing, with an pressure transducer, an antiphase pressure waveform from the transducer drive signal and apply the antiphase pressure waveform to air flowing from the constrictor to the pump to nullify the pulsations in the air; and
measuring, with a measurement device, a target gas in the flow of expired air.

17. The method according to claim 16, wherein the measuring comprises:
launching infrared light through the measurement area using an infrared light source;
filtering the launched infrared light using a bandpass filter having a passband encompassing an absorption line of the target gas; and
detecting the launched and filtered infrared light using a light detector.

18. (canceled).

19. The method according to claim 16, further including:
subtracting, with a comparator the pressure signal from a desired flow rate setpoint signal to generate a flow rate error signal;
amplifying and processing, with a pump controller, the flow rate error signal to generate a pump control signal; and
buffering, with a pump driver, the pump control signal to generate a pump drive signal, and transmit the pump drive signal to the pump.

20. The method according to claim 13, further including:
with the pump driver, increasing the speed of the pump when the pressure signal is less than the desired flow rate setpoint signal; and
with the pump driver, decreasing the speed of the pump when the differential pressure signal is greater than the desired flow rate setpoint signal.
